(19) United States
(12) Patent Application Publication
(10) Pub. No.: US 2024/0293298 A1
(43) Pub. Date: Sep. 5, 2024

(54) COSMETIC AEROSOL PRODUCT

(71) Applicant: Conopco, Inc., d/b/a UNILEVER, Englewood Cliffs, NJ (US)

(72) Inventors: James Peter Jones, London (GB); Louise Ombler McMaster, Leeds (GB)

(21) Appl. No.: 18/591,613

(22) Filed: Feb. 29, 2024

(30) Foreign Application Priority Data
Mar. 1, 2023 (EP) 23159334.4

(51) Int. Cl. A61K 8/26; A61K 8/04; A61K 8/19; A61K 8/31; A61K 8/96; A61Q 15/00

(52) U.S. Cl. CPC A61K 8/26; A61K 8/046; A61K 8/19; A61K 8/31; A61K 8/965; A61Q 15/00; A61K 2800/412; A61K 2800/87

(57) ABSTRACT

A cosmetic aerosol product comprising a pressurised container holding a base composition and a propellant which is a mixture of a compressed gas and a liquefied gas at a weight ratio of from 0.1: 99.9 to 10: 90; wherein the pressurised container comprises:

i). a pressure release valve;

ii). a reservoir containing the base composition and the propellant; and

iii). a dip tube arranged for the transport of the base composition from the reservoir to the pressure release valve;

wherein the base composition comprises a suspension of a particulate solid cosmetic active in a carrier oil, the particulate solid cosmetic active having a concentration of from 5 to 40% by weight in the base composition.

### FIELD OF INVENTION

The present invention relates to aerosol products having a reduced content of liquified propellant, such products being capable of reducing volatile organic carbon (VOC) emissions to the environment.

### BACKGROUND

There are numerous publications describing aerosol products with a reduced content of liquified propellant.

EP 753,561 A1 (P&G, 1997) discloses an aerosol package comprising “ozone friendly” propellants, such as compressed gas or nitrogen, amongst other options.

U.S. Pat. No. 8,191,739B1 (AMREP Inc., 2012) discloses an aerosol dispenser that is pressurised by both compressed gas and liquified gas and which is suitable for foaming products.

WO 2022/012947 A1 (Unilever, 2022) discloses aerosol products having reduced VOC content wherein the propellant comprises an insoluble compressed gas and 5 to 15% by weight of a liquefied gas and wherein the pressure in the container is from 9x105 to 11×105 Pa.

WO 2022/013362 A1 (Unilever, 2022) discloses aerosol hair care products comprising a spraying device having a valve with a restricted tail piece of diameter 0.50 to 0.55 mm and a vapour phase tap of diameter from 0.30 to 0.34 mm.

US 2023/278505 A1 (P&G, 2023) discloses spray devices for antiperspirant or deodorant compositions with a compressed gas propellant.

### SUMMARY OF THE INVENTION

It is an object of the present invention to provide an aerosol product that uses a relatively low level of liquified gas propellant and yet has good sensory and good dispensing properties.

It is a further object of the present invention to provide a cosmetic aerosol product that provides good capture of the spray applied to the target surface.

Products according to the present invention have good sustainability credentials: first, because of their low content of liquified gas, which can often act as greenhouse gases and secondly, because they enable efficient, non-wasteful delivery of the composition to the surface of the human body. Surprisingly, this is enabled with good sensory properties of the spray generated.

The present invention involves the use of a propellant comprising a liquified gas and compressed gas at a relatively narrow ratio range in combination with a cosmetic active suspended in a carrier oil such that the ratio of liquified gas to carrier oil is also selected to be within a relatively narrow range. The combined selected ranges lead to the benefits of the invention as referred to above.

In a first aspect of the invention, there is provided a cosmetic aerosol product comprising a pressurised container holding a base composition and a propellant which is a mixture of a compressed gas and a liquefied gas at a weight ratio of from 0.1: 99.9 to 10: 90; wherein the pressurised container comprises:
i). a pressure release valve;
ii). a reservoir containing all of the base composition and all of the propellant; and
iii). a dip tube arranged for the transport of the base composition from the reservoir to the pressure release valve;
   wherein the base composition comprises a suspension of a particulate solid cosmetic active in a carrier oil, the particulate solid cosmetic active having a concentration of from 5 to 40% by weight in the base composition.

In a second aspect of the invention, there is provided a method of attaining a cosmetic benefit comprising the topical spray application of base composition and propellant mixture as described in the first aspect of the invention.

In a third aspect of the invention, there is provided a method of manufacture of cosmetic aerosol product according to the first aspect of the invention.

Without wishing to be bound by theory, the use of lower amounts of the liquified gas, versus the amount of base composition, may lead to better delivery of the base composition and the particulate solid cosmetic active suspended in the carrier oil as a result of the lower volatility of the combined propellant plus base composition.

The present invention enables excellent deliver of the composition to the surface being sprayed and a highly acceptable particle size for the spray generated, both with a substantially reduced usage of liquified gas.

The weight ratio of nitrogen to liquified gas is from 0.1: 99.9 to 10: 90. Whilst this may seem to be a relatively low content of nitrogen, the pressure generated by the nitrogen may be more than that generated by the liquified propellant at these ratios. This enables much lower levels of liquified gas to be used than is typical in aerosol products currently in the marketplace.

### DETAILED DESCRIPTION

Products according to the invention may comprise compositions suitable for a variety of cosmetic applications. Such compositions include, in particular, antiperspirant compositions.

The balance between the base composition and the propellant is often important to the success of the invention. This ratio is preferably from 30: 70 to 90: 10, more preferably from 40: 60 to 90: 10, and most preferably from 50: 50 to 90: 10. These preferred ratios help the environmental credentials of compositions of the invention and also the delivery and/or sensory benefits attained.

When it is desired to include a vapour phase tap (VPT) in the pressure release valve (vide infra), it is desirable to reduce the highest preferred level for the base to propellant ratio. Hence, in embodiments comprising a VPT, the base to propellant ratio is preferably from 30: 70 to 80: 20, more preferably from 40: 60 to 80: 20, and most preferably from 50: 50 to 80: 20. Reasons for these preferences are as detailed in the paragraph immediately above and also relate to spray quality.

Herein, topical application refers to application to the skin of the human body, in particular to the underarm regions of the human body.

Herein, antiperspirant actives are materials that reduce perspiration on the skin of the human body, typically following topical application.

Herein, the term “spray quality” refers to the extent of droplet breakup achieved on use of the aerosol products. It is desirably to achieve relatively high droplet breakup, such that the spray contains less than 10% of droplets greater than 100 microns in diameter and preferably less than 5% of such droplets.

Herein, methods and uses should be understood to be cosmetic methods/uses, i.e. non-therapeutic methods/uses. Likewise, compositions of the invention are cosmetic compositions, i.e. non-therapeutic.

Herein, all percentages, parts, and ratios are by weight, unless otherwise indicated.

Herein, references to an amount of a material or materials refer to the total amount of material(s) of the type indicated.

Herein, references to an amount of a material are with reference to the total composition of which it is a part, unless otherwise indicated.

Herein, “application” and “applied” relate to application to the skin of the human body, in particular the underarm regions, unless the context dictates otherwise.

Herein, features expressed as “preferred” with regard to a particular aspect of the invention should be understood to be preferred with regard to each aspect of the invention.

Herein, preferred, particularly preferred and especially preferred features of the invention are particularly preferred when used in combination with other preferred, particularly preferred, and especially preferred features of the invention.

Herein, “ambient conditions” refer to 20° C. and 1 atmosphere pressure, unless otherwise indicated.

Herein, indicated pressures should be understood to be “gauge” pressures, i.e. pressures above atmospheric pressure, with the exception of vapour pressures.

Herein, the word “comprising” is intended to mean “including” but not necessarily “consisting of” or “composed of.” In other words, the listed steps or options need not be exhaustive.

Herein, except in the examples or where otherwise explicitly indicated, all numbers in this description and claims indicating amounts of material, physical properties of materials and/or use are to be understood as modified by the word “about”.

Herein, numerical ranges expressed in the format “from x to y” are understood to include x and y. When for a specific feature multiple preferred ranges are described in the format “from x to y”, it is understood that all ranges combining the different endpoints are also contemplated.

Herein, the base composition should be understood to refer to all components of the composition other than the propellant. Components of the base composition include a carrier oil and suspended therein, a particulate solid cosmetic active.

Herein, the carrier oil is an organic material that is a liquid at ambient conditions and is non-miscible with water. I. The carrier oil may be composed of a mixture of component oils.

In preferred embodiments, the carrier oil may also be a masking oil, serving the purpose of reducing visible deposits when the composition accidentally comes into contact with clothing, for example.

The carrier oil and base composition of which it is a part are preferably anhydrous, each containing less than 0.5% free water and preferably less than 0.1% free water. Herein, “free water” is all water excluding that contained as water of hydration in any solid components present in the base compositions.

The carrier oil and base composition of which it is a part are preferably non-ethanolic, each containing less than 0.5% ethanol and preferably less than 0.1% ethanol.

The total amount of carrier oil in the base composition is preferably 35 to 85%, more preferably at least 45% but typically not more than 80% and preferably not more than 75%.

The carrier oil may be selected from any of those known in the art, although hydrophobic carrier oils are preferred.

In some embodiments, preferred carrier oils are silicone oils, that is to say, liquid polyorganosiloxanes. Such materials may be cyclic or linear, examples include Dow Corning silicone fluids 344, 345, 244, 245, 246, 556, and the 200 series; Union Carbide Corporation Silicones 7207 and 7158; and General Electric silicone SF1202.Suitable carrier oils can be selected from alkyl ether oils having a boiling point of above 100° C. and especially above 150° C., including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

Suitable carrier oils can include one or more triglyceride oils. The triglyceride oils commonly comprise the alkyl residues of aliphatic C7 to C20 alcohols, the total number of carbon atoms being selected in conjunction with the extent of olefinic unsaturation and/or branching to enable the triglyceride to be liquid at 20° C. One example is jojoba oil. Particularly preferably, in the triglyceride oil the alkyl residues are linear C18 groups having one, two or three olefinic degrees of unsaturation, two or three being optionally conjugated, many of which are extractable from plants (or their synthetic analogues), including triglycerides of oleic acid, linoleic acid, conjugated linoleic acids, linolenic acid, petroselenic acid, ricinoleic acid, linolenelaidic acid, trans 7-octadecenoic acid, parinaric acid, pinolenic acid, punicic acid and stearidonic acid.

Suitable carrier oils can include those derived from unsaturated C18 acids, including coriander seed oil, impatiens balsimina seed oil, parinarium laurinarium kernel fat oil, sabastiana brasilinensis seed oil, dehydrated castor seed oil, borage seed oil, evening primrose oil, aquilegia vulgaris oil, sunflower (seed) oil and safflower oil. Other suitable oils are obtainable from hemp, and maize corn oil. An especially preferred oil by virtue of its characteristics is sunflower (seed) oil.

Further suitable carrier oils, that can also be emollient oils, comprise alkyl or alkyl-aryl ester oils having a boiling point of above 150° C. (and a melting point of below 20° C.). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C12-15 alkyl benzoate, for example

Finsolv TN™ or Finsolv Sun™

The cosmetic active is a particulate solid material under ambient conditions, suspended in the carrier oil. The cosmetic active is insoluble in the carrier fluid. Any material having a solubility of less than 0.1g/100g under ambient conditions is considered to be insoluble.

The cosmetic active is present as a particulate material, typically obtained by milling a spray-dried aqueous solution of the active. For reasons of reduced nozzle blockage and delivery performance, the cosmetic active preferably has a mean particle size (D50) of less than 100 microns and preferably at least 6 microns. More preferably, the mean particle size is from 6 to 25 microns, and most preferably it is from 7 to 15 microns.

Herein, mean (D50) particle sizes may be measured using (laser) light scattering techniques, for example using a Mastersizer instrument, obtainable from Malvern Instruments. Such instruments are set to produce a volume plot and a lens is selected in accordance with the maker's instructions to accommodate the expected particle size distribution, (or various lenses can be tested until the best lens is identified). Measurements are made by methods known in the art.

Preferred cosmetic actives are antiperspirant actives and preferred antiperspirant actives are astringent aluminium salts, such as aluminium chlorohydrate (ACH) or aluminium sesquichlorohydrate (ASCH).

Herein, ASCH is a particularly preferred active and has the chemical formula Al₂OH_{4.4}Cl_{1.6} to Al₂OH_{4.9}Cl_{1.1}. Most commercial ASCH samples are of chemical formula Al₂OH_{4.7}Cl_{1.3} to Al₂OH_{4.9}Cl_{1.1} and it is preferred to use ASCH salts of this formula.

When employed, preferred antiperspirant actives are “activated” by methods known in the art. Such methods include heating an aqueous solution of the active and then spray-drying and/or treating an aqueous solution of the antiperspirant active with a water-soluble calcium salt and preferably an amino acid, such as glycine.

Particularly preferred antiperspirant actives for use in the present invention are activated ASCH salts, in particular those activated by a water-soluble calcium salt and an amino acid. Such actives are described in EP 2,999,452B1 (Unilever, 2017), EP 3,212,296B1 (Unilever, 2019) and in other publications.

Herein, references to amounts of antiperspirant active refer to the active present as an anhydrous solid, i.e. excluding any water of hydration associated with the active, and also excluding any water-soluble calcium salt or amino acid present in the composition and/or associated with the antiperspirant active.

In preferred embodiments, antiperspirant active is present in the base composition at a level of from 5 to 40%, more preferably from 10 to 40% and most preferably from 15 to 30%.

In certain embodiments of the invention, particularly those in which the cosmetic active is an antiperspirant active, a preferred additional component of the base composition is a suspending agent. Typically suspending agents are clay materials and preferably they are hydrophobically modified clays, otherwise known as organoclay materials. Such materials help keep the particulate solid cosmetic active suspended in the carrier oil.

When employed, particularly preferred suspending agents are bentonite or hectorite clays, especially when they are organoclay materials, i.e., hydrophobically modified and hydrophobically hectorite clays. Especially preferred is disteardimonium hectorite (e.g. Bentone 38V, ex 20 Elementis).

When employed, the suspending agent is typically used at from 0.1 to 1.5%

When a suspending agent is present, in particular a clay-based suspending agent, it is preferred that an activator for the suspending agent is also present. The activator serves to improve the performance of the suspending agent. Preferred activators are triethyl citrate and propylene carbonate. Propylene carbonate is especially preferred.

When employed, the activator is preferably used at a level of from 0.01 to 0.25% of the total composition.

Yet other optional ingredients can include sensory modifiers, such as talc or finely divided polyethylene, such as in an amount of up to 3% by weight of the base composition; colorants, by way of non-limiting example in a proportion of up to 0.5% of the base composition; skin cooling agents such as menthol often selected in an amount of up 0.5%, particularly up to 0.2% of the base composition, and wash-off agents such as non-ionic surfactants, and particularly polyethoxylated fatty alcohols or acids, for example in an amount of up to about 3% of the base composition.

A preferred additional component in compositions used in the invention is a fragrance or fragrance oil, sometimes alternatively called a perfume (oil). The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to.

The fragrance may be present “free”, herein simply termed “fragrance” and/or it may be encapsulated by methods known in the art, in order to delay its release. Often, the delayed release of the fragrance is triggered by contact with moisture or, preferably, by friction. In preferred embodiments, compositions of the invention comprise both free and encapsulated fragrance.

The amount of fragrance oil in the total composition is preferably up to 5%, advantageously is at least 0.5% and particularly from 0.8% to 3.5%.

The amount of fragrance oil in the base composition is preferably up to 20%, advantageously is at least 1% and particularly from 2% to 8%.

The propellant is a mixture of a compressed gas and a liquefied gas at a weight ratio of from 0.1: 99.9 to 10: 90.

Preferred compressed gases are inert, i.e. unreactive materials. A particularly preferred inert compressed gas is nitrogen. Other suitable inert gases are carbon dioxide, helium and argon; although these gases are less preferred for practical reasons, in particular their greater expense.

Prior to first use, the pressure exerted by the total propellant within products according to the invention is preferably from 85 to 125 psig (0.59 to 0.86 MPa) at 25° C., these pressures assisting with good spray quality and delivery.

Prior to first use, the pressure exerted by the compressed gas component of the propellant used in the present invention is preferably from 0.15 to 0.40 MPa) at 25° C., these pressures assisting with the duration of good spray quality and delivery.

Suitable liquified gas propellants for use herein include hydrocarbons, in particular C3-C4 hydrocarbons, and/or hydrofluorocarbons (otherwise known as HFCs), in particular C2-C4 HFCs.

Preferred liquified gas propellants are blends of propane, butane and/or isobutane, with the less preferred addition of an HFC.

When employed, preferred HFCs have from 2 to 4 carbon atoms. A commercially acceptable HFC is HFC-152a, otherwise known as R152a and having the chemical name 1,1-difluroethane (DFE). However, it is preferred that compositions according to the invention are free of HFC in order to minimise greenhouse gas content.

When employed, preferred HFCs have from 2 to 4 carbon atoms. A commercially acceptable HFC is HFC-152a, otherwise known as R152a and having the chemical name 1,1-difluroethane (DFE). However, it is preferred that compositions according to the invention are free of HFC in order to minimise greenhouse gas content.

Other HFCs that may be employed are hydrofluoroolefins (HFOs), such as 1,3,3,3-tetrafluoropropene, otherwise known as HFO-1234ze.

HFCs may be advantageously used in the propellant when a dry sensory feel is desired.

Essential components of hardware used in products according to the invention are:
i). a pressure release valve;
ii). a reservoir containing all of the base composition and all of the propellant; and
iii). a dip tube arranged for the transport of the base composition from the reservoir to the pressure release valve.

The pressurised container holds the aerosol composition under pressure and the pressure release valve releases the composition and allows it to form an aerosol on exit from the container.

The reservoir containing all of the base composition and all of the propellant, i.e. the compressed gas and a liquefied gas, is a single reservoir. The liquefied gas typically equilibrates between the base composition and a gaseous space above the base composition. The compressed gas is typically insoluble in the base composition and sits in the aforementioned gaseous space above the composition. The combination of the two types of propellant in the same reservoir as the base composition enables optimum maintenance of spray capture on first activation. Further, as the contents of the container deplete with repeated use (in particular the propellant which depletes faster than the base), maintenance of good spray rate and delivery is enabled by having the two types of propellant in the same reservoir as the base composition.

Other features typically present within spray devices may also be employed, including:
a valve cup for holding the valve firmly within an outer body of the pressurised container, often used in combination with a valve cup gasket;
a push button for “activating” the pressure release valve; i.e., for opening the valve;
A swirl chamber for enhancing the quality of the spray exiting the pressure release valve. These are sometimes known as “inserts”.

The pressure release valve generally has a valve stem, a body and a spring to aid with closure following activation. A gasket to seal the valve within an associated valve cup is also generally employed. Preferred valve stem orifices have a diameter of from 0.2 to 0.6 mm and more preferably from 0.35 to 0.45 mm.

Other preferred features in the pressure release valve include a vapour phase tap (VPT) and a restricted tail piece (RTP). The VPT enhances the mixing of the propellant and the base composition and helps with spray quality and control of spray rate. Preferred VPTs have a diameter of 0.30 to 0.50 mm, more preferably 0.35 to 0.45 mm.

Herein, references to the “diameter” of a component, such as VPT, RTP or dip tube, refer to its internal diameter.

In some embodiments, it is preferred that the pressure release valve does not have a VPT. Such embodiments typically have a high base to propellant ratio, such as from 75: 25 to 90: 10 or from 80: 20 to 90: 10. The reason for this preference is maintenance of spray quality.

Preferred spray rates for products of the invention are from 0.1 to 0.8 g/s, preferably from 0.2 to 0.6 g/s.

Herein, spray rates are initial spray rates, i.e. spray rates on first actuation of the can and before its contents are significantly depleted.

The RTP helps control the rate of entry of the base composition into the valve; therefore it is also involved in spray quality and spray rate control. Preferably the RTP has a diameter of from 0.5 to 1.0 mm, more preferably from 0.6 to 0.7 mm.

In a preferred aspect of manufacture of products according to the invention, the base composition is prepared first, typically using high shear. The base composition is then placed in the pressurisable can and the compressed gas is added. Finally, the liquified gas is added. A benefit of adding the liquified gas after the compressed gas is that the liquified gas helps to wash back any base composition partially forced out of the can by the temporary pressure release when starting to add the liquified gas. In addition, the pressure build on adding the liquified gas after the compressed gas is much quicker than vice versa.

### Examples

The following Examples are illustrative of the invention claimed and are non-limiting.

TABLE 1

| | | Comparative | |
|---|---|---|---|
| Ingredients | Base | Example 1 | Example 1 |
| Cyclopentasiloxane | 49.10 | 42.00 | 61.08 |
| PPG-14 butyl ether | 16.52 | | |
| Activated ASCH | 27.46 | | |
| Encapsulated | 1.47 | | |
| fragrance | | | |
| Fragrance | 1.90 | | |
| Distearylammonium | 2.75 | | |
| hectorite | | | |
| BHT | 0.44 | | |
| Propylene carbonate | 0.36 | | |
| AP40 | — | — | 38.00 |
| AP21 | — | 20.00 | — |
| HFC-152a | — | 38.00 | — |
| Nitrogen | — | — | 0.92 |
| Total | 100 | 100 | 100 |

The same base formulation, as indicated in Table 1, was used for both Example 1 and Comparative Example 1. Duplicate samples of both Example 1 and Comparative Example 1 were prepared and tested.

All compositions were placed into 210 ml aerosol cans equipped with a pressure release valve having an RTP and a VPT, and a dip tube for transporting the base composition from the reservoir to the pressure release valve. In addition, a standard actuator was used with each sample.

The Example 1 samples had the liquified gas component of the propellant added to the base composition after the compressed gas. Following addition of the compressed gas, the pressure inside the can was 2 bar (0.2 MPa) at 25°° C., this increased on addition of the liquified gas.

The Comparative Example 1 samples were gassed with a mixture of AP21 and HFC-152a at a ratio of 34: 66, the AP21 being a hydrocarbon blend of butane, isobutane and propane (70: 29: 1).

The compositions were sprayed onto pre-weight filter papers (diameter 10 cm) for about 3 secs. and from a distance of 15 cm. The filter papers were weighed again post-spraying to allow the weight of product captured to be calculated. This was divided by the loss of base* from the can (at the time) to give the % Capture.

* calculated as the total loss of weight from can on spraying multiplied by the percentage base in the base-propellant mix. Hence, the difference in base:propellent ratios between the Example samples and the Comparative Example samples is taken into account, giving a legitimate comparison of the spray captures from each. Please note that the base:propellant ratio increased as the % fill in the can decreased, as one might expect.

The spray rate was calculated as weight loss from the can divided by the spray duration.

TABLE 2
Comparative Examples: % Capture vs. % Fill
Comparative Example 1

| | |
|---|---|
| First duplicate | Second duplicate |

| % fill | % base | % capture | % fill | % base | % capture |
|---|---|---|---|---|---|
| 100 | 42 | 54.9 | 100 | 42 | 54.2 |
| 89 | 42 | 57.5 | 90 | 42 | 56.5 |
| 56 | 50 | 54.5 | 54 | 50 | 56.1 |
| 45 | 50 | 57.5 | 45 | 50 | 59.5 |
| 20 | 68 | 53.5 | 20 | 66 | 52.3 |
| 10 | 68 | 61.7 | 10 | 66 | 62.9 |

TABLE 3
Examples: % Capture vs. % Fill
Example 1

| | |
|---|---|
| First duplicate | Second duplicate |

| % fill | % base | % capture | % fill | % base | % capture |
|---|---|---|---|---|---|
| 100 | 61 | 75.7 | 100 | 61 | 73.2 |
| 91 | 61 | 75.0 | 90 | 61 | 81.3 |
| 55 | 67 | 72.1 | 55 | 67 | 77.3 |
| 45 | 67 | 75.3 | 45 | 67 | 79.0 |
| 20 | 76 | 66.1 | 20 | 77 | 72.4 |
| 10 | 76 | 69.6 | 10 | 77 | 76.7 |

The % Capture figures for the Examples according to the invention are significantly higher than those for the Comparative Examples, across the whole lifetime of the product.

TABLE 4
Comparative Examples: Spray Rate vs. % Fill
Comparative Example 1

| First duplicate | | | Second duplicate | | |
|---|---|---|---|---|---|
| % fill | % base | Spray rate (g/s) | % fill | % base | Spray rate (g/s) |
| 100 | 42 | 0.613 | 100 | 42 | 0.628 |
| 89 | 42 | 0.607 | 90 | 42 | 0.620 |
| 56 | 50 | 0.579 | 54 | 50 | 0.565 |
| 45 | 50 | 0.564 | 45 | 50 | 0.588 |
| 20 | 68 | 0.517 | 20 | 66 | 0.553 |
| 10 | 68 | 0.465 | 10 | 66 | 0.498 |

TABLE 5
Examples: Spray Rate vs. % Fill
Example 1

| First duplicate | | | Second duplicate | | |
|---|---|---|---|---|---|
| % fill | % base | Spray rate (g/s) | % fill | % base | Spray rate (g/s) |
| 100 | 61 | 0.648 | 100 | 61 | 0.754 |
| 91 | 61 | 0.549 | 90 | 61 | 0.664 |
| 55 | 67 | 0.446 | 55 | 67 | 0.495 |
| 45 | 67 | 0.407 | 45 | 67 | 0.498 |
| 20 | 76 | 0.377 | 20 | 77 | 0.423 |
| 10 | 76 | 0.339 | 10 | 77 | 0.379 |

The spray rates for the Examples according to the invention do fall away more as the can is depleted, compared with the Comparative Examples, as expected. The unexpected result is that, despite this fact, the deposition of the AP active, represented by aluminium deposited and chloride deposited per second in Tables 6 and 7, is comparable to or higher than that from the Comparative Examples.

TABLE 6
Comparative Examples: Cl and Al Deposited vs. % Fill
Comparative Example 1

| First duplicate | | | | Second duplicate | | | |
|---|---|---|---|---|---|---|---|
| % fill | % base | Al dose | Cl dose | % fill | % base | Al dose | Cl dose |
| 100 | 42 | 7.4 | 8.2 | 100 | 42 | 7.2 | 8.0 |
| 89 | 42 | 7.6 | 8.5 | 90 | 42 | 7.6 | 8.5 |
| 56 | 50 | 8.6 | 9.6 | 54 | 50 | 8.6 | 9.6 |
| 45 | 50 | 8.7 | 9.7 | 45 | 50 | 9.4 | 10.5 |
| 20 | 68 | 10.0 | 11.1 | 20 | 66 | 10.1 | 11.3 |
| 10 | 68 | 10.2 | 11.3 | 10 | 66 | 10.7 | 11.8 |

The chloride (CI) dose in Tables 6 and 7 was measured as part of the study” and the aluminium (Al) dose was calculated from this. Both are expressed in mg/s.

* Chloride dose measurements were performed using a Metrohm Autotitrator using methods known in the art to measure the electrical current produced from chloride ions in the test solutions.

TABLE 7
Examples: Cl and Al Deposited vs. % Fill
Example 1

| First duplicate | | | | Second duplicate | | | |
|---|---|---|---|---|---|---|---|
| % fill | % base | Al dose | Cl dose | % fill | % base | Al dose | Cl dose |
| 100 | 61 | 15.4 | 17.1 | 100 | 61 | 17.5 | 19.5 |
| 91 | 61 | 13.1 | 14.6 | 90 | 61 | 16.5 | 18.3 |
| 55 | 67 | 10.9 | 12.1 | 54 | 67 | 12.7 | 14.1 |
| 45 | 67 | 10.4 | 11.5 | 45 | 67 | 12.9 | 14.3 |
| 20 | 76 | 9.1 | 10.1 | 20 | 77 | 11.7 | 12.9 |
| 10 | 76 | 8.7 | 9.7 | 10 | 77 | 11.2 | 12.4 |

1. A cosmetic aerosol product comprising a pressurised container holding a base composition and a propellant which is a mixture of a compressed gas and a liquefied gas at a weight ratio of from 0.1: 99.9 to 10: 90; wherein the pressurised container comprises:
i). a pressure release valve;
ii). a reservoir containing all of the base composition and all of the propellant; and
iii). a dip tube arranged for the transport of the base composition from the reservoir to the pressure release valve;
wherein the base composition comprises a suspension of a particulate solid cosmetic active in a carrier oil, the particulate solid cosmetic active having a concentration of from 5 to 40% by weight in the base composition.

2. A cosmetic aerosol product according to claim 1, wherein particulate solid cosmetic active is an antiperspirant active.

3. A cosmetic aerosol product according to claim 1 comprising a suspending agent.

4. A cosmetic aerosol product accordingly to claim 3, wherein the suspending agent is a bentonite or hectorite clay.

5. A cosmetic aerosol product accordingly to claim 4 comprising activator for the suspending agent.

6. A cosmetic aerosol product according to claim 1, comprising fragrance at a level of from 0.1 to 5% by weight of the total composition (comprising the base composition and the propellant).

7. A cosmetic aerosol product according to claim 1, wherein the pressure in the container, prior to first use, is from 85 to 125 psig (0.59 to 0.86 MPa) at 25° C.

8. A cosmetic aerosol product accordingly to claim 1, wherein liquified gas is selected hydrocarbons comprising propane, butane, isobutane and mixtures thereof.

9. A cosmetic aerosol product accordingly to claim 1, wherein the pressurised container has an associated valve comprising an RTP of from 0.6 to 0.7 mm and a VPT of from 0.30 to 0.50 mm.

10. A cosmetic aerosol product accordingly to claim 1, wherein the antiperspirant active is calcium-activated.

11. A cosmetic aerosol product accordingly to claim 10, wherein the antiperspirant active is calcium-activated ASCH.

12. A cosmetic aerosol product accordingly to claim 1, wherein the compressed gas is nitrogen.

13. A cosmetic aerosol product accordingly to claim 1, wherein the cosmetic active has a mean particle size is from 6 to 25 microns.

14. A method of delivering a cosmetic composition to the surface of the human body comprising the use of a product according to claim 1.

15. A method of manufacture of a composition according to claim 1, wherein the base composition is prepared first, the base composition is then placed in the pressurisable can and the compressed gas is added; and finally, the liquified gas is then added.
